(19) United States
(12) Patent Application Publication
(10) Pub. No.: US 2008/0096870 A1
(43) Pub. Date: Apr. 24, 2008

(54) Methods and Materials for Treating Mental Illness

(76) Inventors: Anatoly E. Martynyuk, Gainesville, FL (US); Donn M. Dennis, Gainesville, FL (US); Nikolaus Gravenstein, Gainesville, FL (US); Alexander V. Glushakov, Gainesville, FL (US); Viktor Yarotskyy, Gainesville, FL (US); Colin Sumners, Gainesville, FL (US); Christoph Seubert, Gainesville, FL (US)

Correspondence Address: Beusse Wolter Sanks Mora & Maire, 390 N. ORANGE AVENUE, SUITE 2500, ORLANDO, FL, 32801, US

(21) Appl. No.: 11/572,275

(22) PCT Filed: Jul. 18, 2005
(86) PCT No.: PCT/US05/25357

§ 371 Date: Mar. 29, 2007

Related U.S. Application Data
(60) Provisional application No. 60/589,175, filed on Jul. 19, 2004.

(51) Int. Cl. A61K 31/197; A61K 31/451; A61K 31/496; A61K 31/519; A61K 31/5415; A61K 31/551; A61P 25/00; A61P 25/18

(52) U.S. Cl. 514/220; 514/227.5; 514/227.8; 514/252.13; 514/259.41; 514/327; 514/567

(57) ABSTRACT

The subject invention pertains to methods of treating mental illnesses or conditions characterized by a decreased function of NMDA receptors and/or excessively enhanced glutamate release and activity of non-NMDA receptors (AMPA and/or kainate). Specifically disclosed are methods utilizing BrPhe, or isomers of analogs thereof, for treating or preventing mental illness or conditions such as schizophrenia.

### CROSS-REFERENCE TO RELATED APPLICATIONS

This application claims benefit of the Jul. 19, 2004, filing date of U.S. provisional patent application No. 60/589,175.

### BACKGROUND OF THE INVENTION

Mounting evidence suggests that the glutamatergic neurotransmitter system contributes to the pathophysiology of mental illnesses.¹ Schizophrenia, in many ways, is the most severe of the mental illnesses. Schizophrenia is a chronic, severe, and disabling brain disease. Approximately 1 percent of the population develops schizophrenia during their lifetime. More than 2 million Americans suffer from this illness in a given year. The severity of the symptoms and long-lasting, chronic pattern of schizophrenia often cause a high degree of disability.²⁻⁴

Antipsychotic drugs are the best treatment now available, but they do not “cure” schizophrenia or ensure that there will be no further psychotic episodes. They may even produce side effects that further complicate treatment. During the early phases of drug treatment, patients may be troubled by side effects such as drowsiness, restlessness, muscle spasms, tremor, dry mouth, or blurring of vision. The long-term side effects of antipsychotic drugs may pose a considerably more serious problem. For example, tardive dyskinesia (TD) is a disorder characterized by involuntary movements most often affecting the mouth, lips, and tongue, and sometimes the trunk or other parts of the body such as arms and legs.⁵⁻⁶ It may persist despite withdrawal of the offending antipsychotic drug.

There is growing evidence that glutamatergic dysfunction is involved in the pathophysiology of schizophrenia.⁷ It was recently proposed that psychotic symptoms are produced by a disturbed balance between the pre- and postsynaptic parts of a glutamatergic synapse; in particular, due to a decreased function of NMDA receptors and excessively enhanced glutamate transmission at non-NMDA receptors (AMPA and/or kainate). This overactivation of AMPA/kainite receptors is thought to cause cognitive dysfunction.⁸⁻¹⁰ Therefore, now there is a consensus among researchers that in order to be effective in treatment of schizophrenia, therapeutic agents should either enhance NMDA receptor function or reduce the excess release of glutamate and/or block postsynaptic AMPA/kainate receptors.¹¹ The inventors have discovered a compound that combines unique properties. The halogenated derivatives of L-Phe, 3,5-dibromo-L-Phe and 3-bromo-L-Phe, augment NMDA receptor-mediated current, significantly depresses glutamate release and AMPA/kainate receptor function.

### BRIEF SUMMARY OF THE INVENTION

The subject invention concerns methods for treating a mental illness or condition which comprises administering 3,5-dibromo-L-phenylalanine, 3-bromo-L-phenylalanine, or isomers and analogs thereof. In a specific aspect, the invention is related to treatment of mental illnesses or conditions characterized by decreased function of NMDA receptors and/or enhanced glutamate release or activity of non-NMDA (AMPA and/or kainate) glutamatergic receptors.

The present invention also concerns methods for modulating NMDA and non-NMDA receptor activity and glutamate release.

### BRIEF DESCRIPTION OF THE DRAWINGS

FIG. 1. 3,5-dibromo-L-phenylalanine (3,5-DBr-L-Phe) activates NMDA receptor-mediated currents in rat cerebrocortical neurons in concentration-dependent maimer. A: Example of NMDA receptor mediated fluctuating background currents recorded from the single neuron in the presence of different concentrations of 3,5-DBr-L-Phe. Horizontal bars denote 3,5-DBr-L-Phe applications. NMDA receptor mediated currents were recorded in TTX-containing (0.3 μM), Mg²⁺-free extracellular solution at holding membrane potential of −60 mV. NBQX (10 μM), strychnine (1 μM) and picrotoxin (100 μM) were added to the extracellular solution to block AMPA/kainate, glycine and GABA receptors, respectively. B and C: Concentration-response relationships for 3,5-DBr-L-Phe to activate total NMDA receptor-mediated current (I_{3,5-DBr-L-Phe}) and fluctuating background currents, respectively. Amplitude of total NMDA receptor current was calculated by subtracting mean value of the current in the absence of 3,5-DBr-L-Phe from the current recorded in the presence of 3,5-DBr-L-Phe and plotted against the concentration of 3,5-DBr-L-Phe. NMDA receptor-mediated background noise current was calculated as standard deviation of mean. Data expressed as mean±S.E.M. for 5-14 cells. *, P<0.01 compared to control.

FIG. 2. Properties of 3,5-dibromo-L-phenylalanine (3,5-DBr-L-Phe)-activated current. A and B: Activating effect of 3,5-DBr-L-Phe on NMDA receptor-mediated current does not depend on concentration of glycine. Example of the effect of 3,5-DBr-L-Phe (100 μM) on NMDA receptor-mediated background current recorded from the single neuron in the presence of different concentrations of glycine (A). Horizontal bars denote 3,5-DBr-L-Phe (100 μM) and glycine applications. Histograms summarizing the effect of 3,5-DBr-L-Phe on amplitude of NMDA receptor-mediated currents in the presence of different concentrations of glycine are depicted in panel B. C and D: Activating effect of 3,5-DBr-L-Phe on NMDA receptor-mediated current depends on concentration of NMDA in extracellular solution. C: Examples of NMDA (3, 10 and 30 μM) activated currents (I_{NMDA}) recorded from the same neuron exposed to 3,5-DBr-L-Phe (100 μM). 3,5-DBr-L-Phe exposure was initiated 45 s before the start of NMDA application. D: Effect of 3,5-DBr-L-Phe (100 μM) on current activated by NMDA (3, 10, 30, 100 and 1000 μM) in the presence of two concentrations of glycine (0.1 μM and 10 μM). Amplitude of total I_{NMDA} was normalized to control values (I_{NMDA} in the absence of 3,5-DBr-L-Phe) and plotted against the concentration of NMDA. The total I_{NMDA} was measured as a sum of the current activated by 3,5-DBr-L-Phe without NMDA (steady state inward current) and of the current recorded in the presence of 3,5-DBr-L-Phe and NMDA. Data expressed as mean±S.E.M. for 3-5 cells. *, P<0.01 compared to control. E: 3,5-DBr-L-Phe-activated current is blocked by NMDA receptor specific antagonists. Representative example of depression of 3,5-DBr-L-Phe-activated current by NMDA receptor antagonist AP-5. Horizontal bars denote 3,5-DBr-L-Phe (100 μM) and AP-5 (20 μM) applications. Similar results were obtained from total of 6 neurons. NMDA receptor-mediated background currents were recorded at the same conditions as described in FIG. 1A.

FIG. 3. 3,5-dibromo-L-phenylalanine (3,5-DBr-L-Phe) depresses AMPA/kainate receptor-mediated mEPSCs in rat cerebrocortical cultured neurons in concentration-dependent manner. A: Representative traces of AMPA-kainate mEPSCs recorded from a cortical neuron under the following conditions: control; in the presence of 3,5-DBr-L-Phe (100 μM); after washout of 3,5-DBr-L-Phe. AMPA/kainate receptor-mediated currents were recorded in TTX-containing (0.3 μM) extracellular solution at holding membrane potential of −60 mV. MK-801 (10 μM), strychnine (1 μM) and picrotoxin (100 μM) were added to the extracellular solution to block NMDA, glycine and GABA receptors, respectively. B and C: Concentration-response relationships for 3,5-DBr-L-Phe to attenuate AMPA/kainate receptor-mediated mEPSC frequency and amplitude, respectively. Data was normalized to control values and plotted against the concentration of 3,5-DBr-L-Phe. Data is expressed as mean±SEM of 6-7 cells. Intervention vs. Control: *, P<0.01. Curve fitting and estimation of value of IC₅₀ for the frequency of AMPA/kainate mEPSCs was made according to the 4-parameter logistic equation. The IC₅₀ for the effect of 3,5-DBr-L-Phe on the amplitude of AMPA/kainate mEPSCs was not determined because the small number of mEPSCs in the presence of 3,5-DBr-L-Phe concentrations higher than 100 μM made it impossible to adequately determine the average amplitude of non-NMDAR-mediated mEPSCs.

FIG. 4. 3,5-dibromo-L-phenylalanine (3,5-DBr-L-Phe) causes depression of glutamate release and activity of postsynaptic AMPA-kainate receptors. A: Effect of 3,5-DBr-L-Phe on the evoked EPSCs in rat cerebrocortical cultured neuron. Examples of average EPSCs (20 traces average) in control conditions (open circle), in 3,5-DBr-L-Phe (filled circle). Synaptic responses were evoked by applying two sub-threshold electric stimuli (0.4-1 ms, 50-90 V, 250 ms apart) to an extracellular electrode (a patch electrode filled with the extracellular solution) positioned in the vicinity of the presynaptic neuron. Sweeps were recorded at 10 s intervals. After 20 sweeps, 100 μM 3,5-DBr-L-Phe was added. Neuron was held in whole-cell mode at Vₕ=−60 mV in Mg²⁺ (1 mM) containing extracellular solution. Strychnine (1 μM) and picrotoxin (100 μM) were added to the extracellular solution to block glycine and GABA receptors, respectively. B: Values of the 2nd/1st amplitude ratio of the paired EPSC responses. The amplitude of the 1st and 2nd EPSCs were measured against the baseline; each point represents an average of five subsequent sweeps. Data expressed as mean±S.E.M. for 7 cells. *, P<0.01 compared to control.

C and D: 3,5-DBr-L-Phe depresses AMPA-activated currents (I_{AMPA}) in rat cerebrocortical cultured neurons. Examples of AMPA-activated currents recorded from the same rat cortical neuron before application of 3,5-DBr-L-Phe, during exposure to different concentrations of BrPhe (noted in figure) and after washout of 3,5-DBr-L-Phe (C). 3,5-DBr-L-Phe exposure was initiated 45 s before the start of AMPA application. Horizontal bar denotes AMPA (3 μM) application. Peak I_{AMPA} was normalized to control values (in the absence of 3,5-DBr-L-Phe) and plotted against the concentration of 3,5-DBr-L-Phe (D). Data expressed as mean±S.E.M. for 3-5 cells. *, P<0.01 compared to control.

FIG. 5. 3,5-DBr-L-Phe does not significantly affect gamma-aminobutyric (GABA) receptor-mediated mIPSCs and elicited action potentials in rat cerebrocortical cultured neurons. A: Representative GABA receptor-mediated mIPSCs recorded from the same neuron before (control), during (100 μM), and after (wash) application of 3,5-DBr-L-Phe. GABA receptor-mediated mIPSCs were recorded in TTX-containing (0.3 μM) extracellular solution at holding membrane potential of −60 mV. NBQX (10 μM), MK-801 (10 μM) and strychnine (1 μM) were added to the extracellular solution to block AMPA/kainate, NMDA and glycine receptors, respectively. B: Histograms summarizing the effects of 3,5-DBr-L-Phe (100 μM) on the amplitude and frequency of GABA receptor-mediated mIPSCs. Summary data is expressed as mean±SEM of 5 cells.

C: Examples of action potentials elicited by depolarizing the membrane with inward current pulses of 2 ms duration and 2 nA amplitude in control (before application of 3,5-DBr-L-Phe), in the presence of 3,5-DBr-L-Phe (100 μM) and after wash-out of the drug. Similar responses were recorded from 5 of 5 neurons.

FIGS. 6-8 show formulas representing analogs of the NMDA receptor enhancing compounds of the subject invention.

### DETAILED DISCLOSURE OF THE INVENTION

The subject invention is based on the inventors discovery that a compound having both the ability to enhance NMDA function, while preferably inhibiting glutamate release and activity of non-NMDA glutamatergic receptors would be desired for treating mental illnesses such as schizophrenia. The subject invention is directed methods for treating a mental illness or condition which is related to, or which can be affected by, modulation of NMDA and/or non-NMDA (AMPA and/or kainite) receptor activity and glutamate release. The treatment methods as described herein can be either prophylactic in nature, curative in nature, or serve to alleviate symptoms of such mental illness or condition.

Particularly, the subject invention concerns methods for treating mental illnesses or conditions characterized by decreased function of NMDA receptors. In a specific embodiment, the subject invention concerns methods for treating mental illnesses or conditions characterized by a decrease in function of NMDA receptors coupled with a potentiation of glutamate release and activity of non-NMDA receptors. Target mental illnesses and conditions of the subject methods include, but are not limited to, schizophrenia, delirium, anxiety, depression, stress, dementia, psychosis, mania and bipolar effective disorder. In an alternative embodiment, the methods target mental ailments characterized by undesired dopaminergic transmission. Without being held to any specific mechanism, it is the inventors belief, that since dopamine is derived from L-Phenylalanine, that halogen substituted forms of L-Phenylalanine, will result in less dopamine being generated and/or block of dopamine receptors, and therefore less dopaminergic transmission.

Unless otherwise indicated, as used herein, the term “BrPhe” as used herein, including the claims, refers to 3,5-dibromo-L-Phenylalanine and 3-bromo-L-Phenylalanine, isomers thereof, including optical isomers (e.g., dextrorotatory (D-), levorotatory (L-), or mixtures thereof (DL-)), and analogs thereof. Accordingly, the use of BrPhe in the claims includes analogs and isomers of 3,5-dibromo-L-Phenylalanine and 3-bromo-L-Phenylalanine. Mixtures of 3,5-dibromo-L-Phenylalanine, with its isomer, or with analogs, or with 3-bromo-L-Phenylalanine, or with naturally occurring aromatic amino acids, and their isomers or analogs, are also contemplated. See U.S. Pat. No. 6,620,850 for disclosure of aromatic amino acids. Analogs of 3,5-dibromo-L-phenylalanine include, but are not limited to 3,5-dibromo-L-Tyrosine and 3,5-dibromo-L-Tryptophan.

The subject invention is at least partly based on the observation that BrPhe is capable of enhancing function of NMDA receptors while having an inhibitory affect on glutamate release and non-NMDA receptor function.

Analogs of BrPhe can be substituted at various positions. FIGS. 6-8 show formulas representing analogs of 3,5-dibromo-L-Phenylalanine, 3,5-dibromo-L-Tyrosine, and 5,7-dibromo-L-Tryptophan, respectively. It should be understood that while these 3,5 dibromo substituted aromatic amino acids can be produced by modifying the naturally occurring aromatic amino acids (phenylalanine, tryptophan, and tyrosine), it is contemplated that other starting materials (e.g., other amino acids) can be utilized to produce the 3,5 dibromo substituted analogs of the subject invention, using methods of organic synthesis known to those skilled in the art.

Referring now to each of the formulas in FIGS. 6 through 8, R¹ and R², which may be the same or different, can be H, hydroxyl (OH), alkyl, alkenyl, alkynyl, halogen, or alkoxy. For 3,5-dibromo-L-Phenylalanine, are both bromine. For analogs of 3,5-dibromo-L-Phenylalanine, one of R¹ or R², or both, should be a halogen. For analogs of 3-bromo-L-Phenylalanine, either R¹ and R² are bromine. Typically bromine is the halogen, but bromine may be optionally substituted with other halogens. R³ can be H, OH, 0, alkyl, alkenyl, alkynyl, halogen, or alkoxy. R⁴ can be H, OH, alky, alkenyl, alkynyl, halogen, or alkoxy, but is not present when R³ is O. R⁵ can be H, alkyl, alkenyl, alkynyl, halogen, or alkoxy.

In one embodiment, in the formulas shown in FIG. 6 and FIG. 8, the pair of substituents, R³ and R⁴, can together form a cyclic group, wherein the resulting ring structure is selected from the group consisting of cycloalkyl, cycloalkenyl, heterocycloalkyl, heterocycloalkenyl, aryl, and heteroaryl. The resulting ring structure can optionally be benzofused at any available position.

As used in the specification, the term “alkyl” refers to a straight or branched chain alkyl moiety. In one embodiment, the alkyl moiety is C₁₋₈ alkyl, which refers to an alkyl moiety having from one to eight carbon atoms, including for example, methyl, ethyl, propyl, isopropyl, butyl, tert-butyl, pentyl, hexyl, octyl, and the like. In another embodiment, the alkyl moiety is C₁₋₃ alkyl.

The term “alkenyl” refers to a straight or branched chain alkyl moiety having in addition one or more carbon—carbon double bonds, of either E or Z stereochemistry where applicable. In one embodiment, the alkenyl moiety is C₂₋₆ alkenyl, which refers to an alkenyl moiety having two to six carbon atoms. This term would include, for example, vinyl, 1-propenyl, 1- and 2-butenyl, 2-methyl-2-propenyl, and the like.

The term “alkynyl” refers to a straight or branched chain alkyl moiety having in addition one or more carbon—carbon triple bonds. In one embodiment, the alkynyl moiety is C₂₋₆ alkynyl, which refers to an alkynyl moiety having two to six carbon atoms. This term would include, for example, ethynyl, 1-propynyl, 1- and 2-butynyl, 1-methyl-2-butynyl, and the like.

The term “alkoxy” refers to an alkyl-O-group, in which the alky group is as previously described.

The term “halogen” refers to fluorine, chlorine, bromine, or iodine.

The term “cycloalkenyl” refers to an alicyclic moiety having from three to six carbon atoms and having in addition one double bond. This term includes, for example, cyclopentenyl and cyclohexenyl.

The term “heterocycloalkyl” refers to a saturated heterocyclic moiety having from two to six carbon atoms and one or more heteroatom from the group N, O, S (or oxidized versions thereof) which may be optionally benzofused at any available position. This includes for example azetidinyl, pyrrolidinyl, tetrahydrofuranyl, piperidinyl, benzodioxole and the like.

The term “heterocycloalkenyl” refers to an alicyclic moiety having from three to six carbon atoms and one or more heteroatoms from the group N, O, S and having in addition one double bond. This term includes, for example, dihydropyranyl.

The term “aryl” refers to an aromatic carbocyclic ring, optionally substituted with, or fused with, an aryl group. This term includes, for example phenyl or naphthyl.

The term “heteroaryl” refers to aromatic ring systems of five to ten atoms of which at least one atom is selected from O, N, and S, and optionally substituted with an aryl group substituent. This term includes for example furanyl, thiophenyl, pyridyl, indolyl, quinolyl and the like.

The term “aryl group substituent” refers to a substituent chosen from halogen, CN, CF₃, CH₂ F, and NO₂.

The term “benzofused” refers to the addition of a ring system sharing a common bond with the benzene ring.

The term “cycloimidyl” refers to a saturated ring of five to ten atoms containing the atom sequence —C(═O)NC(═O)—. The ring may be optionally benzofused at any available position. Examples include succinimidoyl, phthalimidoyl and hydantoinyl.

The term “optionally substituted” means optionally substituted with one or more of the groups specified, at any available position or positions.

It will be appreciated that BrPhe analogs according to the invention can contain one or more asymmetrically substituted carbon atoms (i.e., chiral centers). The presence of one or more of these asymmetric centers in an analog of the formulas shown in FIGS. 6-8 can give rise to stereoisomers, and in each case the invention is to be understood to extend to all such stereoisomers, including enantiomers and diastereomers, and mixtures including racemic mixtures thereof.

Isomers and analogs can be used according to the subject invention so long as the isomers or analogs exhibit the desired biological activity. Biological activity characteristics can be evaluated, for example, through the use of binding assays, or assays that measure cellular response.

An isomer or analog having the capability to modulate NMDA and non-NMDA activity would be considered to have the desired biological activity in accordance with the subject invention. More preferably, the BrPhe, or isomers and analogs thereof, have the ability to enhance NMDA receptor function and decrease non-NMDA glutamatergic receptor function. Most, preferably, the BrPhe, or isomers and analogs thereof, have the ability to enhance NMDA receptor function, decrease non-NMDA glutamatergic receptor function, and attenuate glutamate release. For therapeutic applications, an isomer or analog of the subject invention preferably has the capability to enhance activity of NMDA receptors and inhibit activity of non-NMDA receptors.

According to the methods of the subject invention, BrPhe is administered in an amount effective to deliver BrPhe to the brain. For example, BrPhe can be administered in an amount sufficient to bring the patient's blood plasma BrPhe level within the range of about 10 μM to about 1000 μM. Preferably, the patient's blood plasma BrPhe level is brought to within the range of about 10 μM to about 1000 μM. More preferably, the patient's blood plasma BrPhe level is brought to within the range of about 10 μM to about 500 μM. However, the appropriate concentration of BrPhe in the blood for treatment of mental illnesses and conditions can be adjusted, as the permeability of the blood-brain barrier can vary markedly with different disease states. In addition, the precise dosage will depend on a number of clinical factors, for example, the type of patient (e.g., human, non-human mammal, or other animal), age of the patient, and the condition under treatment and its severity. A person having ordinary skill in the art would readily be able to determine, without undue experimentation, the appropriate dosages required to achieve the appropriate levels.

In another embodiment, the methods of the subject invention comprise co-administering a facilitating substance that can enhance uptake of BrPhe across the blood-brain barrier, thereby more efficiently raising the concentration of the BrPhe within the brain, and/or increases the activity of the BrPhe that is already present in the brain (e.g., endogenously or exogenously present). As used herein, the term “co-administering” means including the facilitating substance within a composition that also comprises BrPhe, or separately administering the facilitating substance before, during, or after administration of BrPhe. Examples of facilitating substances include, but are not limited to, agents that enhance BrPhe transport. Alterations in barrier function, including modulation of barrier permeability, have been demonstrated through the activation of second messenger pathways. For example, stimulation of the protein kinase C (PKC) pathway is reported to increase barrier permeability, including the transport of amino acids across the blood-brain barrier (Ermisch et al., 1988; Rubin et al., 1999; Lynch et al., [1990]. Lynch J J, Ferro T J, Blumenstock F A, Brockenauer A M, Malik A B. 1990. Increased endothelial albumin permeability mediated by protein kinase-C activation. J Clin Invest 85: 1991-1998. Rubin L L, Staddon J M. 1999. The cell biology of the blood-brain barrier. Annu Rev Neurosci 22: 11-28. Ermisch A, Landgraf R, Brust P, Kretzschmar R, Hess J. 1988. Peptide receptors of the cerebral capillary endothelium and the transport of amino acids across the blood-brain barrier. In: Rakic L, Begley D J, Davson H, Zlokovic B V, editors. Peptide and amino acid transport mechanisms in the central nervous system. London: Macmillan. p 51-54. Since P-glycoprotein in the BBB restricts the brain entry of many drugs, inhibition of this drug transporter may be an option for improved drug delivery to brain. (Kemper E M, Boogerd W, Thuis I, Beijnen J H, Van Tellingen O. Modulation of the blood-brain barrier in oncology: therapeutic opportunities for the treatment of brain tumours? Cancer Treat Rev. 2004; 30: 415-23.) Grant G A, Meno J R, Nguyen T S, Stanness K A, Janigro D, Winn R H. J Adenosine-induced modulation of excitatory amino acid transport across isolated brain arterioles. Neurosurg. 2003; 98: 554-60. Bartus R T, Elliott P J, Dean R L, Hayward N J, Nagle T L, Huff M R, Snodgrass P A, Blunt D G. Controlled modulation of BBB permeability using the bradykinin agonist, RMP-7. Exp Neurol. 1996;142:14-28.

According to another embodiment, the present invention is directed to combination therapy that comprises the concomitant, simultaneous or sequential administration of BrPhe and at least one neuroleptic agent that include, but not limited to, clozapine, haloperidol, olanzapine, risperidone, flupenthixol, chliorpromazine, thioridazine, trifluoperzine, and zuclopenthixol, to enhance their therapeutic effects.

A “patient” refers to a human, non-human mammal, or other animal in which modulation of NMDA receptors and/or glutamate release and non-NMDA receptors will have a beneficial effect. Patients in need of treatment involving modulation of such receptors can be identified using standard techniques known to those in the medical profession.

A further aspect of the present invention provides a method of modulating the activity of an NMDA receptor and/or non-NMDA receptors and glutamate release, and includes the step of contacting the receptor with BrPhe that modulates one or more activities of the receptor, in general, either stimulating activity or inhibiting activity of the receptor. The method can be carried out in vivo or in vitro. The contacting step can be carried out with the receptor at various levels of isolation. For example, the BrPhe can be placed in contact with the receptor while the receptor is associated with tissue, the cell (e.g. neurons or glia), or fully isolated.

High blood concentrations of L-Phe (>1200 μM versus 55-60 μM in healthy patients) cause the neurological disease phenylketonuria (PKU) (Knox WE [1972] Stanbury J B et al., eds., 3^{rd} ed., McGraw Hill, New York, pp. 266-295; Scriver C R et al. [1989] Scriver et al., eds., McGraw-Hill, New York, pp. 495-546). Unless diagnosed and treated early in life with a L-Phe-restricted diet, irreversible brain damage occurs (Berry H K et al. [1979] Dev Med Child Neurol 21:311-320; Pennington B F et al. [1985] Am J Ment Defic 89:467-474). However, high concentrations of L-Phe are harmful only during the first years of life, and only during chronic exposure to elevated concentrations of this amino acid. Phenylketonuric patients typically discontinue their therapeutic special diet when they reach adulthood. All PKU-related studies converge on the same conclusion that after the age of 10 years, IQ development is stable for different degrees of dietary relaxation (Burgard P [2000] Eur J Pediatr 159 (Suppl 2): S74-S79). Importantly, BrPhe augments NMDA receptor function, whereas L-Phe has opposite, depressant, effect on NMDA receptor activity (see ref. 12 and 13). Therefore, BrPhe may also have beneficial effect for the PKU patients.

While BrPhe can be administered as an isolated compound, it is preferred to administer BrPhe in the form of a pharmaceutical composition. The subject invention thus further provides pharmaceutical compositions comprising BrPhe as an active ingredient, or physiologically acceptable salt(s) thereof, in association with at least one pharmaceutically acceptable carrier or diluent. The pharmaceutical composition can be adapted for various forms of parenteral administration, such as intravenous and nasal routes. Administration can be continuous or at distinct intervals as can be determined by a person skilled in the art.

The pharmaceutical compounds of the subject invention can be formulated according to known methods for preparing pharmaceutically useful compositions. Formulations are described in a number of sources which are well known and readily available to those skilled in the art. For example, Remington's Pharmaceutical Sciencse (Martin E W [1995] Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The subject invention also provides an article of manufacture useful in treating a mental illness characterized by decreased function of NMDA receptors. The article contains a pharmaceutical composition containing an BrPhe, and a pharmaceutically acceptable carrier or diluent. The article of manufacture can be, for example, an intravenous bag, a syringe, a nasal applicator, or a microdialysis probe. The article of manufacture can also include printed material disclosing instructions for the parenteral treatment of the neurological condition. The printed material can be embossed or imprinted on the article of manufacture and indicate the amount or concentration of the BrPhe, recommended doses for parenteral treatment of the neurological condition, or recommended weights of individuals to be treated.

The compounds are preferably formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, to produce blood plasma BrPhe levels to greater than about 10 μM.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

The term “average blood plasma BrPhe level(s)” as used herein refers to an average of BrPhe concentration of a patient maintained over a period of time. Average blood plasma BrPhe level(s) can be determined empirically and established by a patient parameter, such as weight, or can be determined on a patient by patient basis by taking two or more readings of BrPhe levels obtained from said patient. The two or more readings may be taken within hours of each other. Preferably, the two or more readings are obtained at least a week from each other.

The term “regimen” as used herein refers to an administration of two or more dosages sequentially spaced in time so as to maintain average blood plasma levels of BrPhe at a predetermined level. The space in time is preferably 3 or more hours.

In addition, the compounds may be formulated as the sole pharmaceutically active ingredient in the composition or may be combined with other active ingredients. Liposomal suspensions, including tissue-targeted liposomes, particularly tumor-targeted liposomes, may also be suitable as pharmaceutically acceptable carriers. These may be prepared according to methods known to those skilled in the art. For example, liposome formulations may be prepared as described in U.S. Pat. No. 4,522,811.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo systems (see, e.g., Rosenthal et al. (1996) Antimicrob. Agents Chemother. 40(7):1600-1603; Dominguez et al. (1997) J. Med. Chem. 40:2726-2732; Clark et al. (1994) Molec. Biochem. Parasitol. 17:129; Ring et al. (1993) Proc. Natl. Acad. Sci. USA 90:3583-3587; Engel et al. (1998) J. Exp. Med. 188(4):725-734; Li et al. (1995) J. Med. Chem. 38:5031) and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. Typically a therapeutically effective dosage should produce a serum concentration of active ingredient of from about 0.1 ng/ml to about 50-100 μ/ml. The pharmaceutical compositions typically should provide a dosage of from about 0.001 mg to about 2000 mg of compound per kilo-gram of body weight per day. Pharmaceutical dosage unit forms are prepared to provide from about 1 mg to about 1000 mg and preferably from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Preferred pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets are presently preferred. The compositions are in liquid, semi-liquid or solid form and are formulated in a manner suitable for each route of administration. Preferred modes of administration include parenteral and oral modes of administration. Oral administration is presently most preferred.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, preferably 0.1-85%, typically 75-95%.

The active compounds or pharmaceutically acceptable derivatives may be prepared with carriers that protect the compound against rapid elimination from the body, such as time release formulations or coatings.
1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.
2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.
3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, preferably 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.
4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.
5. Compositions for Other Routes of Administration

Other routes of administration, such as transdermal patches and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.
6. Articles of Manufacture

The compounds or pharmaceutically acceptable derivatives may be packaged as articles of manufacture containing packaging material, a compound or pharmaceutically acceptable derivative thereof provided herein, which is comprises BrPhe.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,352. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. In a preferred embodiment, the article of manufacture comprises indicia on its surface indicating it contains of BrPhe, and even more preferably, indicating the concentration of BrPhe.

### EXAMPLE 1

Methods

Neuronal Cultures. Cerebral cortices were dissected from newborn rats and treated with 0.25% trypsin to dissociate the cells. Dissociated cells were resuspended in Dulbecco's Modified Eagle's Medium (DMEM) containing 10% plasma derived horse serum (PDHS) and were plated in poly-L-lysine-coated, 35 mm Nunc plastic tissue culture dishes (3.0×10⁶ cells/dish/2ml media). Cultures were maintained in an atmosphere of 5% CO₂/95% air.

Electrophysiological recordings: Voltage- and current-clamp recordings of membrane ionic currents and potentials were conducted by using Axopatch 200B and Axoclamp 1B amplifiers (Axon Instruments, Foster City, Calif.). The perforated nystatin- and gramicidin-based patch-clamp recording techniques were used to reduce nonspecific rundown of intracellular processes. Neurons were used for electrophysiological recordings between 12 and 27 days in vitro. During the experiment, if the neuron showed either a marked change in holding current or a noticeable alteration in amplitude or shape of the capacitance transients, the data from that neuron was discarded. Patch microelectrodes were pulled from 1.5 mm borosilicate glass tubing using a two-stage vertical pipette puller (Narishige, East Meadow, N.Y.). When filled with recording solution, patch microelectrodes had a resistance of 3-5 MΩ. For rapid application of agonist-containing solutions to neurons, the SF-77B system (Warner Instrument Corp., Hamden, Conn.) was used.

The miniature EPSCs were recorded in TTX-containing (0.3-1 μM), Mg²⁺-free (in case of NMDA receptor recording) extracellular solution at Vh=−60 mV. In order to isolate the NMDA component of GluR-mediated EPSCs, the non-NMDAR (AMPA/kainate) antagonist NBQX (10-20 μM) was added to extracellular solutions. To isolate the non-NMDAR-mediated EPSCs, the experiments were performed in the presence of NMDAR channel blocker, MK-801 (5-10 μM), or in the presence of the NMDAR antagonist, AP-5 (20 μM). Strychnine (1 μM) and picrotoxin (20 μM) were added to the extracellular solution to block glycine and GABA receptors, respectively. Our previous experiments showed that further addition of the non-NMDAR antagonist, NBQX (10 μM), completely abolished all postsynaptic currents, indicating that the recorded mEPSCs were mediated through activation of a non-NMDAR (AMPA/kainate) subtype of GluRs. The basic extracellular solution contained (in mM): NaCl 140, KCl 4, CaCl₂ 2, MgCl₂ 1,4-(2-hydroxyethyl)-1-piperazineethanesulfonic acid (HEPES) 10, and glucose 11. The pH of the extracellular solution was adjusted to 7.4 using NaOH. The main solution for filling the patch electrodes contained (in mM): Cs gluconate 135, NaCl 5, KCl 10, MgCl₂ 1, CaCl₂ 1, EGTA 11, HEPES 10, Na₂ATP 2, Na₂GTP 0.2 nM. The pH of the intracellular solution was adjusted to 7.4 using CsOH. To record GABAR-mediated miniature inhibitory postsynaptic currents (mIPSCs), picrotoxin (100 μM) in the extracellular solution and Cs gluconate (135 mM) in the intrapipette solution were replaced with NBQX (5 μM) and KCl (135 mM), respectively. Various concentrations of NMDA, AMPA, 3,5-DBr-L-Phe, glycine were added to the extracellular solution according to the protocols described. All compounds were purchased from Sigma Chemical Co., St Louis, Mo.

The digitized data was analyzed off-line using the Mini-Analysis Program (Synaptosoft, Leonia, N.J.) or the pCLAMP9 (Axon Instruments) (Axon Instruments, Union City, Calif.). Miniature EPSCs were identified and confirmed by analyzing the rise time, decay time, and waveform of each individual spontaneous event.

General data analysis. Values are reported as mean ± SEM. Prior to parametric testing, the assumption of normality was validated using the Kolmogorov-Smirnov test with Lilliefor's correction (SSPS v10, SPSS, Inc., Chicago, Ill.). Multiple comparisons among groups were analyzed using ANOVA (two or one way repeated measures with 2 or 1 way replication where appropriate) followed by Student-Newman-Keuls testing. Single comparisons were analyzed using a 2-tailed Student's t test. A P<0.05 was considered significant.

Results

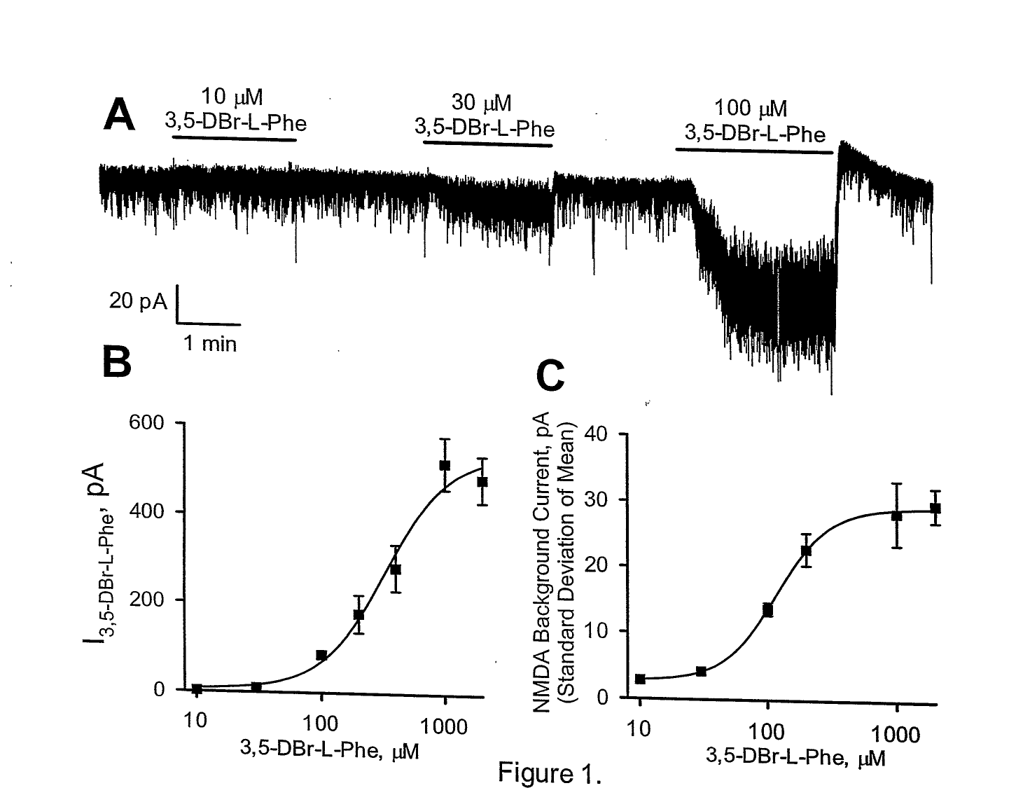

FIG. 1. 3,5-dibromo-L-phenylalanine (3,5-DBr-L-Phe) activates NMDA receptor-mediated currents in rat cerebrocortical neurons in concentration-dependent manner. A: Example of NMDA receptor mediated fluctuating background currents recorded from the single neuron in the presence of different concentrations of 3,5-DBr-L-Phe. Horizontal bars denote 3,5-DBr-L-Phe applications. NMDA receptor mediated currents were recorded in TTX-containing (0.3 μM), Mg²⁺-free extracellular solution at holding membrane potential of −60 mV. NBQX (10 μM), strychnine (1 μM) and bicuculline (20 μM) were added to the extracellular solution to block AMPA/kainate, glycine and GABA receptors, respectively. B and C: Concentration-response relationships for 3,5-DBr-L-Phe to activate total NMDA receptor-mediated current (I_{3,5-DBr-L-Phe}) and fluctuating background currents, respectively. Amplitude of total NMDA receptor current was calculated by subtracting mean value of the current in the absence of 3,5-DBr-L-Phe from the current recorded in the presence of 3,5-DBr-L-Phe and plotted against the concentration of 3,5-DBr-L-Phe. NMDA receptor-mediated background noise current was calculated as standard deviation of mean. Data expressed as mean±S.E.M. for 5-14 cells. *, P<0.01 compared to control.

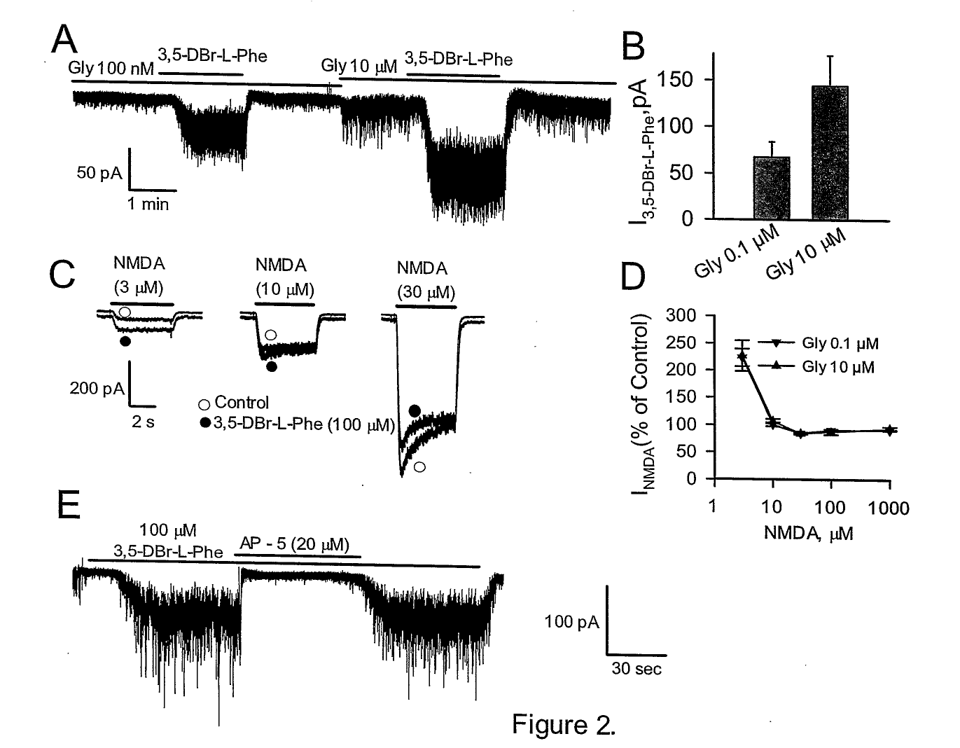

FIG. 2. Properties of 3,5-dibromo-L-phenylalanine (3,5-DBr-L-Phe)-activated current. A and B: Activating effect of 3,5-DBr-L-Phe on NMDA receptor-mediated current does not depend on concentration of glycine. Example of the effect of 3,5-DBr-L-Phe (100 μM) on NMDA receptor-mediated background current recorded from the single neuron in the presence of different concentrations of glycine (A). Horizontal bars denote 3,5-DBr-L-Phe (100 μM) and glycine applications. Histograms summarizing the effect of 3,5-DBr-L-Phe on amplitude of NMDA receptor-mediated currents in the presence of different concentrations of glycine are depicted in panel B. C and D: Activating effect of 3,5-DBr-L-Phe on NMDA receptor-mediated current depends on concentration of NMDA in extracellular solution. C: Examples of NMDA (3, 10 and 30 μM) activated currents (I_{NMDA}) recorded from the same neuron exposed to 3,5-DBr-L-Phe (100 μM). 3,5-DBr-L-Phe exposure was initiated 45 s before the start of NMDA application. D: Effect of 3,5-DBr-L-Phe (100 μM) on current activated by NMDA (3, 10, 30, 100 and 1000 μM) in the presence of two concentrations of glycine (0.1 μM and 10 μM). Amplitude of total I_{NMDA} was normalized to control values (I_{NMDA} in the absence of 3,5-DBr-L-Phe) and plotted against the concentration of NMDA. The total I_{NMDA} was measured as a sum of the current activated by 3,5-DBr-L-Phe without NMDA (steady state inward current) and of the current recorded in the presence of 3,5-DBr-L-Phe and NMDA. Data expressed as mean±S.E.M. for 3-5 cells. *, P<0.01 compared to control. E: 3,5-DBr-L-Phe-activated current is blocked by NMDA receptor specific antagonists. Representative example of depression of 3,5-DBr-L-Phe-activated current by NMDA receptor antagonist AP-5. Horizontal bars denote 3,5-DBr-L-Phe (100 μM) and AP-5 (20 μM) applications. Similar results were obtained from total of 6 neurons. NMDA receptor-mediated background currents were recorded at the same conditions as described in FIG. 1A.

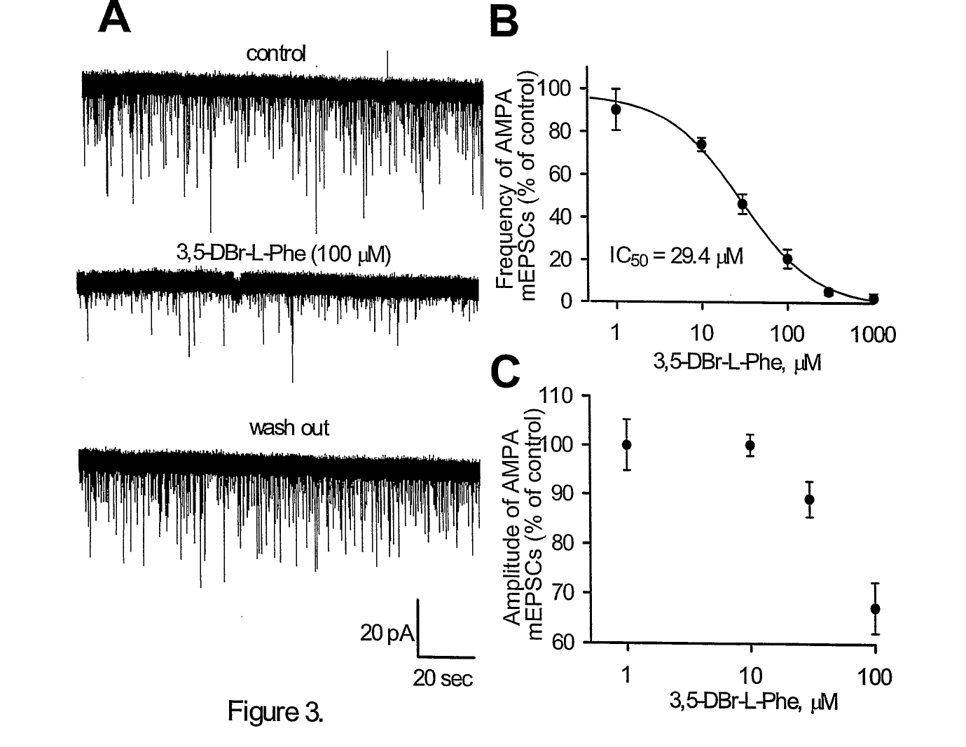

FIG. 3. 3,5-dibromo-L-phenylalanine (3,5-DBr-L-Phe) depresses AMPA/kainate receptor-mediated mEPSCs in rat cerebrocortical cultured neurons in concentration-dependent manner. A: Representative traces of AMPA-kainate mEPSCs recorded from a cortical neuron under the following conditions: control; in the presence of 3,5-DBr-L-Phe (100 μM); after washout of 3,5-DBr-L-Phe. AMPA/kainate receptor-mediated currents were recorded in TTX-containing (0.3 μM) extracellular solution at holding membrane potential of −60 mV. MK-801 (10 μM), strychnine (1 μM) and picrotoxin (100 μM) were added to the extracellular solution to block NMDA, glycine and GABA receptors, respectively. B and C: Concentration-response relationships for 3,5-DBr-L-Phe to attenuate AMPA/kainate receptor-mediated mEPSC frequency and amplitude, respectively. Data was nonnalized to control values and plotted against the concentration of 3,5-DBr-L-Phe. Data is expressed as mean±SEM of 6-7 cells. Intervention vs. Control: *, P<0.01. Curve fitting and estimation of value of IC₅₀ for the frequency of AMPA/kainate mEPSCs was made according to the 4-parameter logistic equation. The IC₅₀ for the effect of 3,5-DBr-L-Phe on the amplitude of AMPA/kainate nEPSCs was not determined because the small number of mEPSCs in the presence of 3,5-DBr-L-Phe concentrations higher than 100 μM made it impossible to adequately determine the average amplitude of non-NMDAR-mediated nEPSCs.

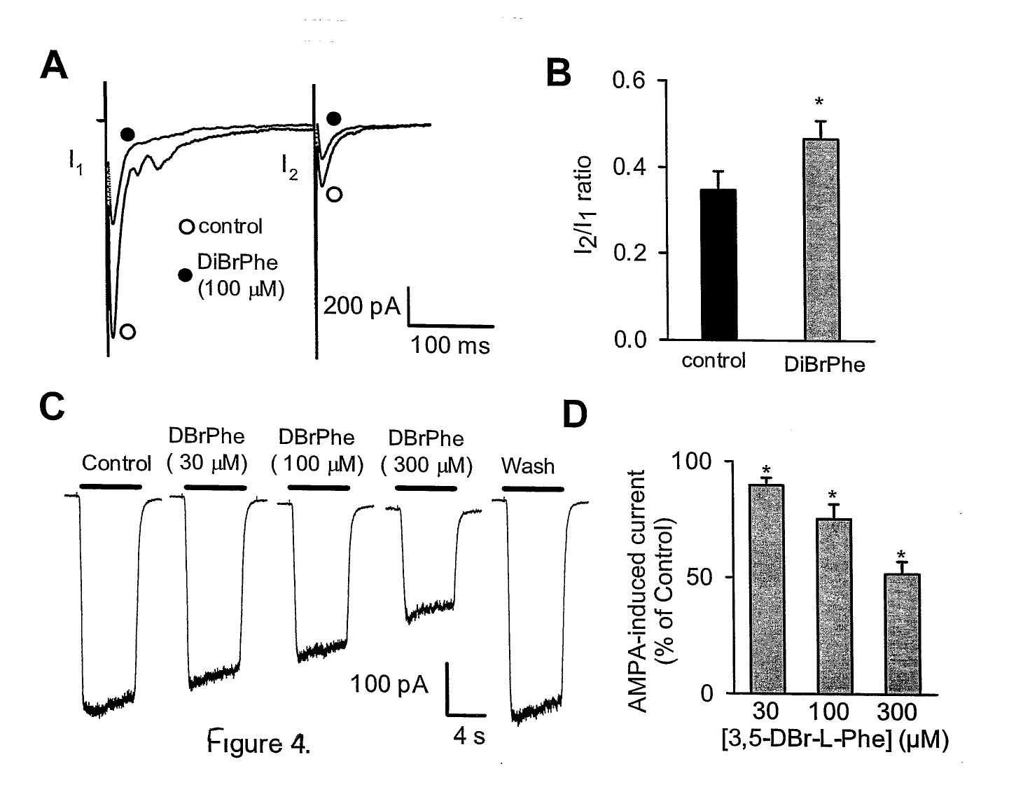

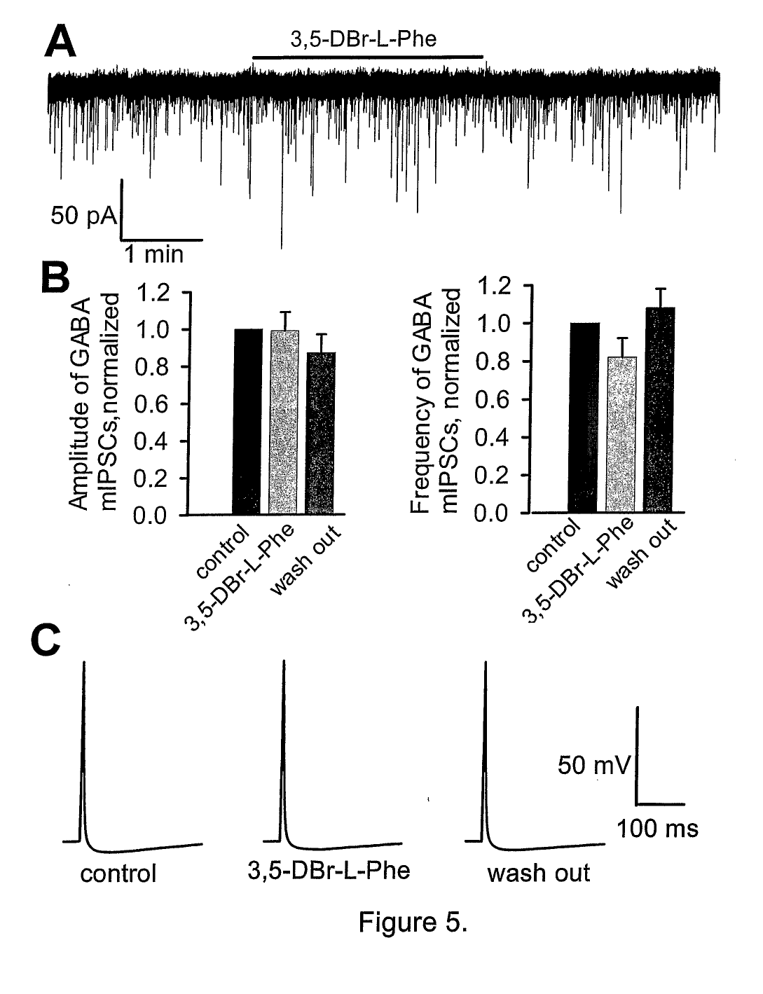

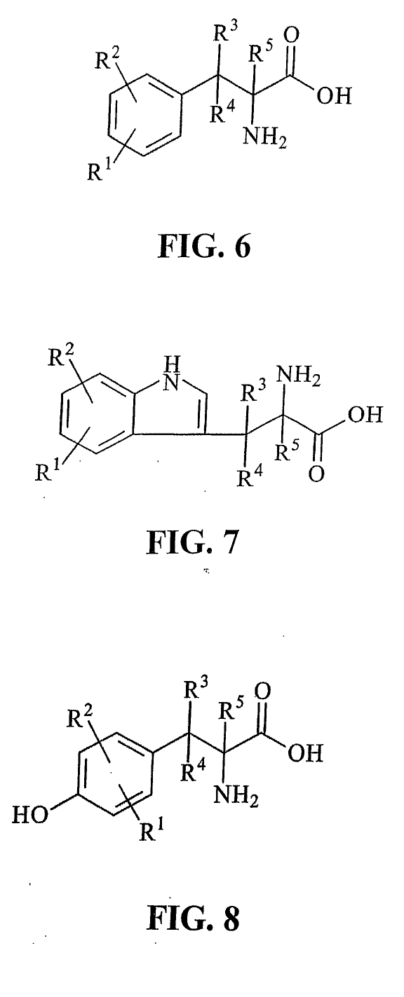

FIG. 4. 3,5-dibromo-L-phenylalanine (3,5-DBr-L-Phe) causes depression of glutamate release and activity of postsynaptic AMPA-kainate receptors. A: Effect of 3,5-DBr-L-Phe on the evoked EPSCs in rat cerebrocortical cultured neuron. Examples of average EPSCs (20 traces average) in control conditions (open circle), in 3,5-DBr-L-Phe (filled circle). Synaptic responses were evoked by applying two sub-threshold electric stimuli (0.4-1 ms, 50-90 V, 250 ms apart) to an extracellular electrode (a patch electrode filled with the extracellular solution) positioned in the vicinity of the presynaptic neuron. Sweeps were recorded at 10 s intervals. After 20 sweeps, 100 μM 3,5-DBr-L-Phe was added. Neuron was held in whole-cell mode at Vₕ=−60 mV in Mg²⁺ (1 mM) containing extracellular solution. Strychnine (1 μM) and picrotoxin (100 μM) were added to the extracellular solution to block glycine and GABA receptors, respectively. B: Values of the 2nd/1st amplitude ratio of the paired EPSC responses. The amplitude of the 1st and 2nd EPSCs were measured against the baseline; each point represents an average of five subsequent sweeps. Data expressed as mean±S.E.M. for 7 cells. *, P<0.01 compared to control.

C and D: 3,5-DBr-L-Phe depresses AMPA-activated currents (I_{AMPA}) in rat cerebrocortical cultured neurons. Examples of AMPA-activated currents recorded from the same rat cortical neuron before application of 3,5-DBr-L-Phe, during exposure to different concentrations of BrPhe (noted in figure) and after washout of 3,5-DBr-L-Phe (C). 3,5-DBr-L-Phe exposure was initiated 45 s before the start of AMPA application. Horizontal bar denotes AMPA (3 μM) application. Peak I_{AMPA} was normalized to control values (in the absence of 3,5-DBr-L-Phe) and plotted against the concentration of 3,5-DBr-L-Phe (D). Data expressed as mean±S.E.M. for 3-5 cells. *, P<0.01 compared to control.

FIG. 5. 3,5-DBr-L-Phe does not significantly affect gamma-aminobutyric (GABA) receptor-mediated mIPSCs and elicited action potentials in rat cerebrocortical cultured neurons. A: Representative GABA receptor-mediated mIPSCs recorded from the same neuron before (control), during (100 μM), and after (wash) application of 3,5-DBr-L-Phe. GABA receptor-mediated mIPSCs were recorded in TTX-containing (0.3 μM) extracellular solution at holding membrane potential of −60 mV. NBQX (10 μM), MK-801 (10 μM) and strychnine (1 μM) were added to the extracellular solution to block AMPA/kainate, NMDA and glycine receptors, respectively. B: Histograms summarizing the effects of 3,5-DBr-L-Phe (100 μM) on the amplitude and frequency of GABA receptor-mediated mIPSCs. Summary data is expressed as mean±SEM of 5 cells.

C: Examples of action potentials elicited by depolarizing the membrane with inward current pulses of 2 ms duration and 2 nA amplitude in control (before application of 3,5-DBr-L-Phe), in the presence of 3,5-DBr-L-Phe (100 μM) and after wash-out of the drug. Similar responses were recorded from 5 of 5 neurons.

### REFERENCES

1. Krystal J H, Sanacora G, Blumberg H, Anand A, Charney D S, Marek G, Epperson C N, Goddard A, Mason G F. Glutamate and GABA systems as targets for novel antidepressant and mood-stabilizing treatments. Mol. Psychiatry 2002; 7: S71-80.
2. Schizophrenia. National Institute of Mental Health. http://www.nimh.nih.gov/publicat/schizoph.cfm
3. Siever L J, Davis K L. The pathophysiology of schizophrenia disorders: perspectives from the spectrum. Am J Psychiatry. 2004; 161: 398-413.
4. Freedman R. Schizophrenia. N Engl J Med. 2003; 349: 1738-49.
5. Correll C U, Leucht S, Kane J M. Lower risk for tardive dyskinesia associated with second-generation antipsychotics: a systematic review of 1-year studies. Am J Psychiatry. 2004; 161: 414-25.
6. Serretti A, De Ronchi D, Lorenzi C, Berardi D. New antipsychotics and schizophrenia: a review on efficacy and side effects. Curr Med Chem. 2004; 11: 343-58.
7. Miyamoto S, LaMantia A S, Duncan G E, Sullivan P, Gilmore J H, Lieberman J A. Recent advances in the neurobiology of schizophrenia. Mol Intervent. 2003; 3: 27-39.
8. Moghaddam B, Adams B W. Reversal of phencyclidine effects by a group II metabotropic glutamate receptor agonist in rats. Science. 1998; 281: 1349-52.
9. Moghaddam B, Adams B, Verma A, Daly D. Activation of glutamatergic neurotransmission by ketamine: a novel step in the pathway from NMDA receptor blockade to dopaminergic and cognitive disruptions associated with the prefrontal cortex. J Neurosci. 1997; 17: 2921-7.
10. Olney J W, Newcomer J W, Farber N B. NMDA receptor hypofunction model of schizophrenia. J Psychiatr Res. 1999; 33: 523-33.
11. Holden C. Excited by glutamate. Science. 2003; 300; 1866-1868.
12. Glushakov A V, Dennis D M, Morey T E, Sumners C, Cucchiara R F, Seubert C N, Martynyuk A E. Specific inhibition of N-methyl-D-aspartate receptor function in rat hippocampal neurons by L-phenylalanine at concentrations observed during phenylketonuria. Mol Psychiatry. 2002; 7: 359-67.
13. Glushakov A V, Dennis D M, Sumners C, Seubert C N, Martynyuk A E. L-phenylalanine selectively depresses currents at glutamatergic excitatory synapses. J Neurosci Res. 2003; 72: 116-24.

All patents, patent applications, and publications referred to or cited herein are incorporated by reference in their entirety to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

What is claimed is:

1. A method of treating mental illness or condition characterized by a decreased function of NMDA receptors, excessively enhanced glutamate release or activity of non-NMDA glutamatergic receptors, or combinations thereof, comprising administering an effective amount of BrPhe to a patient in need thereof.

2. The method of claim 1, wherein said mental illness is schizophrenia.

3. The method according to claim 1, wherein BrPhe is administered to the patient orally, intranasally, or intravenously.

4. The method according to claim 1, wherein BrPhe thereof, is administered in an amount sufficient to raise the patient's blood plasma BrPhe level to within a range of about 10 μM to about 2000 μM.

5. The method according to claim 1, wherein BrPhe is administered in an amount sufficient to raise the patient's blood plasma BrPhe level to within a range of about 10 μM to about 1000 μM.

6. The method according to claim 1, wherein BrPhe is administered in an amount sufficient to raise the patient's blood plasma BrPhe level to within a range of about 10 μM to about 1000 μM.

7. The method of claim 1, wherein BrPhe is administered according to a regimen to produce an average blood plasma BrPhe level to within a range of about 10 μM to about 1000 μM of BrPhe over a period of at least one week.

8. The method of claim 1, wherein BrPhe is administered according to a regimen to produce an average blood plasma BrPhe level to within a range of about 10 μM to about 1000 μM of BrPhe over a period of at least two weeks.

9. The method of claim 1, wherein BrPhe is administered according to a regimen to produce an average blood plasma BrPhe level to within a range of about 10 μM to about 1000 μM of BrPhe over a period of at least 4 weeks.

10. The method of claim 1, wherein BrPhe is administered according to a regimen to produce an average blood plasma level of about 10 μM to about 1000 μM of BrPhe over a period of at least two months.

11. The method of claim 1, wherein BrPhe is administered according to a regimen to produce an average blood plasma level of about 10 μM to about 1000 μM of BrPhe over a period of at least six months.

12. A method of treating a mental illness or condition comprising administering an effective amount of BrPhe to a patient in need thereof, wherein said mental illness is post-anesthesia delirium, anxiety, depression, stress, dementia, psychosis, mania, and bipolar effective disorder.

13. The method according to claim 12, wherein BrPhe is administered to the patient orally, intranasally, or intravenously.

14. The method according to claim 12, wherein BrPhe is administered in an amount sufficient to raise the patient's blood plasma BrPhe level to within a range of about 20 μM to about 2000 μM.

15. The method according to claim 12, wherein the BrPhe is administered in an amount sufficient to raise the patient's blood plasma BrPhe level to within a range of about 10 μM to about 1800 μM.

16. The method according to claim 12, wherein BrPhe is administered in an amount sufficient to raise the patient's blood plasma BrPhe level to within a range of about 10 μM to about 1500 μM.

17. The method of claim 12, wherein BrPhe is administered according to a regimen to produce an average blood plasma BrPhe level to within a range of about 10 μM to about 1000 μM over a period of at least one week.

18. The method of claim 12, wherein BrPhe is administered according to a regimen to produce an average blood plasma BrPhe level to within a range of about 10 μM to about 1000 μM over a period of at least two weeks.

19. The method of claim 12, wherein BrPheis administered according to a regimen to produce an average blood plasma BrPhe level to within a range of about 10 μM to about 1000 μM over a period of at least 4 weeks.

20. The method of claim 11, wherein BrPhe is administered according to a regimen to produce an average blood plasma BrPhe level to within a range of about 10 μM to about 1000 μM over a period of at least two months.

21. The method of claim 12, wherein BrPhe is administered according to a regimen to produce an average blood plasma BrPhe level to within a range of about 10 μM to about 1000 μM over a period of at least six months.

22. A method of treating a mental illness or condition of a patient comprising: diagnosing whether said patient suffers from a mental illness or condition; and administering a dosage of BrPhe sufficient to lessen symptoms of said mental illness or condition.

23. A combination therapy for treating a patient suffering from a mental illness or condition, said therapy comprising the administration concomitantly, simultaneously or sequentially, of therapeutically effective amounts of BrPhe and at least one neuroleptic agent selected from the group consisting of clozapine, haloperidol, olanzapine, risperidone, flupenthixol, chlorpromazine, thioridazine, trifluoperzine, and zuclopenthixol.
